(19) United States
(12) Patent Application Publication
(10) Pub. No.: US 2026/0355390 A1
(43) Pub. Date: Nov. 12, 2026

(54) NERVE REPAIR DEVICE

(71) Applicant: Arthrex, Inc., Naples, FL (US)

(72) Inventors: Joshua William Hustedt, Scottsdale, AZ (US); Iliana Gonzalez, Bonita Springs, FL (US)

(73) Assignee: Arthrex, Inc., Naples, FL (US)

(21) Appl. No.: 19/652,432

(22) Filed: Apr. 20, 2026

Related U.S. Application Data
(60) Provisional application No. 63/800,615, filed on May 6, 2025.

(51) Int. Cl. A61L 27/54; A61L 27/36

(52) U.S. Cl. CPC A61L 27/54; A61L 27/3604; A61L 27/3683; A61L 2430/32

(57) ABSTRACT

Provided herein are nerve repair devices comprising a nerve conduit having a proximal end, a distal end, an exterior surface, and an interior surface defining a lumen, the lumen comprising decellularized human placental connective tissue matrix (DHPCTM). The lumen can further comprise adipose tissue (such as micronized adipose tissue) and/or bone marrow aspirate. Also provided are methods of repairing nerves, methods of making nerve repair devices, and kits for nerve repair.

### PRIORITY

This application claims the benefit of U.S. Ser. No. 63/800,615, filed on May 6, 2025, which is incorporated by reference in its entirety.

### BACKGROUND

Nerve graft devices having a combination of neurogenic, neuroinductive, and neuroconductive properties are needed in the art. Nerve graft devices currently on the market do not have all of these properties. Furthermore, nerve allografts are expensive and need to be stored frozen, while the use of nerve autografts can result in significant nerve morbidity.

### SUMMARY

Provided herein are nerve repair devices comprising a nerve conduit having a proximal end, a distal end, an exterior surface, and an interior surface defining a lumen, the lumen comprising decellularized human placental connective tissue matrix (DHPCTM). The lumen of the nerve repair device can further comprise adipose tissue, bone marrow aspirate, or both adipose tissue and bone marrow aspirate. The adipose tissue can be micronized adipose tissue.

The lumen can further comprise nerve autograft or allograft material. The micronized adipose tissue and/or bone marrow aspirate can be autograft micronized adipose tissue and/or autograft bone marrow aspirate. The micronized adipose tissue can comprise particles of 0.3 mm to 1.0 mm. The DHPCTM can comprise placental extracellular matrix, Type I collagen, Type III collagen, and Type IV collagen, elastin, and glycosaminoglycans. The nerve conduit can be comprised of one or more of collagen, poly(lactic-co-glycolic acid) (PLGA), silk, polytetrafluoroethylene (PTFE), polydimethylsiloxane (PDMS), silicone, a salicylic acid-based polymer, a salicylsalicylic acid-based polymer, a difluorophenyl-salicylic acid-based polymer, collagen, fibrin, fibronectin, alginate, gelatin, keratin, thrombin, poly-glycolic acid, poly-(lactic acid), poly-caprolactone, poly-(L-lactic acid), poly(3-hydroxybutyric acid), poly((bis(hydroxyethyl) terephthalate-ethyl phosphoester/terephthaloyl chloride), poly(2-hydroxyethyl methacrylate) (PHEMA), a co-polymer of PHEMA and methyl methacrylate, and poly(acrylonitrile-co-methylacrylate). The nerve conduit can be comprised of bovine collagen. The nerve conduit can have an interior diameter of 0.2 mm to 2 cm and a length of 1 mm to 2,000 mm. The lumen can further comprise insulin-like growth factor 1, insulin-like growth factor 2, PDGF-A, PDGF-B, PDGF-C, PDGF-D), fibroblast growth factor (FGF)1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, Keratinocyte growth factor-2, transforming growth factor-α, transforming growth factor-β, brain derived neurotrophic factor, ciliary neurotrophic factor, leukemia inhibitory factor, interleukin-6, oncostatin M, prolactin, neurturin, glial cell line-derived neurotrophic factor, Schwann cells, endothelial cells, fibroblasts, genetically altered cells, or combinations thereof. The nerve repair device can have neurogenic, neuroinductive, and/or neuroconductive properties.

Another aspect provides methods of in vivo, in vitro, and ex vivo nerve repair. The methods can comprise placing a proximal end of a nerve conduit in contact with a first portion of the nerve and a distal end of the nerve conduit in contact with a second portion of the nerve such that the first and second portions of the nerve are each positioned in contact with the distal and proximal ends or within a lumen at each of the distal and proximal ends. The nerve conduit can comprise a proximal end, a distal end, an exterior surface, and an interior surface defining a lumen, the lumen comprising decellularized human placental connective tissue matrix (DHPCTM). The lumen of the nerve conduit can further comprise adipose tissue, bone marrow aspirate, or both adipose tissue and bone marrow aspirate. The adipose tissue can be micronized adipose tissue.

The first portion of the nerve can be secured to the proximal end of the nerve conduit and the second portion of the nerve can be secured to the distal end of the nerve conduit. The first portion of the nerve and the second portion of the nerve can be opposite ends of a fully or partially transected nerve. The first portion of the nerve and the second portion of the nerve can be proximal and distal portions of an injured but still intact nerve. The nerve can be a sensory nerve, a motor nerve, or a mixed nerve. The nerve can be from a mouse, rat, dog, cat, pig, sheep, goat, camel, cow, horse, non-human primate, or human. In an aspect, placing the proximal end of the nerve conduit of the nerve device in contact with a first portion of the nerve and the distal end of the nerve conduit in contact with a second portion of the nerve can comprise loading the nerve conduit into a needle or catheter and injecting the nerve conduit into a tissue. The lumen can further comprise nerve autograft material, nerve, allograft material, insulin-like growth factor 1, insulin-like growth factor 2, PDGF-A, PDGF-B, PDGF-C, PDGF-D), fibroblast growth factor (FGF)1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, Keratinocyte growth factor-2, transforming growth factor-α, transforming growth factor-β, brain derived neurotrophic factor, ciliary neurotrophic factor, leukemia inhibitory factor, interleukin-6, oncostatin M, prolactin, neurturin, glial cell line-derived neurotrophic factor, Schwann cells, endothelial cells, fibroblasts, genetically altered cells, or combinations thereof. The methods can further comprise contacting the nerve repair device with fibrin adhesive, autologous, thrombin-enhanced serum, or a combination thereof. The nerve repair device can have neurogenic, neuroinductive, and/or neuroconductive properties.

Another aspect provides methods of nerve repair in a subject. The methods can comprise harvesting adipose tissue and/or bone marrow aspirate from the subject and mixing the adipose tissue and/or bone marrow aspirate with DHPCTM to form a mixture. The mixture can be placed into a lumen of a nerve conduit having a proximal end, a distal end, an exterior surface, and an interior surface defining the lumen. The proximal end of the nerve conduit can be placed in contact with a first portion of a nerve and the distal end of the nerve conduit can be placed in contact with a second portion of the nerve such that the first and second portions of the nerve are each positioned in contact with the distal and proximal ends of the nerve conduit or within the lumen at each of the distal and proximal ends of the nerve conduit. The adipose tissue can be micronized and washed prior to use. The lumen can further comprise nerve autograft material, nerve, allograft material, insulin-like growth factor 1, insulin-like growth factor 2, PDGF-A, PDGF-B, PDGF-C, PDGF-D), fibroblast growth factor (FGF)1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, Keratinocyte growth factor-2, transforming growth factor-α, transforming growth factor-β, brain derived neurotrophic factor, ciliary neurotrophic factor, leukemia inhibitory factor, interleukin-6, oncostatin M, prolactin, neurturin, glial cell line-derived neurotrophic factor, Schwann cells, endothelial cells, fibroblasts, genetically altered cells, or combinations thereof. The method can further comprising contacting the nerve repair device with fibrin adhesive, or autologous, thrombin-enhanced serum, or a combination thereof. The nerve repair device can have neurogenic, neuroinductive, and/or neuroconductive properties.

Yet another aspect provides methods of preparing a nerve repair device. The methods can comprise harvesting adipose tissue and/or bone marrow aspirate from a subject and mixing the adipose tissue and/or bone marrow aspirate with DHPCTM to form a mixture and placing the mixture into a lumen of a nerve conduit having a proximal end, a distal end, an exterior surface, and an interior surface defining the lumen such that a filled nerve conduit is formed. The adipose tissue can be micronized and washed prior to use. The lumen can further comprise nerve autograft material, nerve, allograft material, insulin-like growth factor 1, insulin-like growth factor 2, PDGF-A, PDGF-B, PDGF-C, PDGF-D), fibroblast growth factor (FGF)1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, Keratinocyte growth factor-2, transforming growth factor-α, transforming growth factor-β, brain derived neurotrophic factor, ciliary neurotrophic factor, leukemia inhibitory factor, interleukin-6, oncostatin M, prolactin, neurturin, glial cell line-derived neurotrophic factor, Schwann cells, endothelial cells, fibroblasts, genetically altered cells, or combinations thereof.

Even another aspect provides a kit comprising decellularized human placental connective tissue matrix (DHPCTM); and a nerve conduit having a proximal end, a distal end, an exterior surface, and an interior surface defining the lumen. The kit can further comprise a system for preparing micronized adipose tissue and/or bone marrow aspirate and a container to mix the micronized adipose tissue and/or bone marrow aspirate with the DHPCTM. The kit can further comprise an insertion catheter for filling the nerve conduit. The kit can further comprise sutures and suture needles. The kit can further comprise nerve autograft material, nerve, allograft material, insulin-like growth factor 1, insulin-like growth factor 2, PDGF-A, PDGF-B, PDGF-C, PDGF-D), fibroblast growth factor (FGF)1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, Keratinocyte growth factor-2, transforming growth factor-α, transforming growth factor-β, brain derived neurotrophic factor, ciliary neurotrophic factor, leukemia inhibitory factor, interleukin-6, oncostatin M, prolactin, neurturin, glial cell line-derived neurotrophic factor, Schwann cells, endothelial cells, fibroblasts, genetically altered cells, or combinations thereof. The kit can additionally comprise fibrin adhesive. The kit can also comprise a system for producing autologous, thrombin-enhanced serum.

### DETAILED DESCRIPTION

Provided herein are nerve repair devices having neurogenic, neuroinductive, and/or neuroconductive properties. In an aspect, a nerve repair device comprises a nerve conduit having decellularized human placental connective tissue matrix within the lumen of the nerve conduit. In an aspect, the lumen can comprise additional materials such as adipose tissue and/or bone marrow aspirate as described herein.

### Nerve Repair Conduit

A nerve repair conduit includes a proximal end, a distal end, an exterior surface, and an interior surface that defines an interior lumen. A nerve repair conduit can be comprised of any suitable material including, for example, one or more of collagen (e.g., bovine, equine, canine, porcine, human, etc.) poly(lactic-co-glycolic acid) (PLGA), silk, polytetrafluoroethylene (PTFE), polydimethylsiloxane (PDMS), silicone, a salicylic acid-based polymer, a salicylsalicylic acid-based polymer, a difluorophenyl-salicylic acid-based polymer, collagen, fibrin, fibronectin, alginate, gelatin, keratin, thrombin, poly-glycolic acid, poly-(lactic acid), poly-caprolactone, poly-(L-lactic acid), poly(3-hydroxybutyric acid), poly((bis(hydroxyethyl) terephthalate-ethyl phosphoester/terephthaloyl chloride), poly(2-hydroxyethyl methacrylate) (PHEMA), a co-polymer of PHEMA and methyl methacrylate, or poly(acrylonitrile-co-methylacrylate).

The interior lumen can have a diameter of about 0.1 mm to about 2 cm, e.g., about 0.1, 0.25, 0.5, 0.75, 1.0, 1.25, 1.5, 1.75, 2.0, 3.0, 4.0, 5.0, 10, 25, 50, 75, 100, 250, 500, 750, 1,000, 1,250, 1,500, 1,750, 2,000 or more mm. In an aspect, an interior lumen can be about 2,000, 1,750, 1,500, 1,250, 1,000, 750, 500, 250, 100, 75, 50, 25, 10, 5.0, 4.0, 3.0, 2.0, 1.75, 1.5, 1.25, 1.0, 0.75, 0.5, 0.25, 0.1 mm or less.

A nerve conduit can have a thickness (e.g., thickness between interior surface and exterior surface) of about 0.05 mm to about 3 mm (e.g., about 0.05, 0.1, 1.0, 1.5, 2.0, 2.5, 3.0 mm or more).

A nerve conduit can be about 1.0, 1.25, 1.5, 1.75, 2.0, 3.0, 4.0, 5.0, 10, 25, 50, 75, 100, 250, 500, 750, 1,000, 1,250, 1,500, 1,750, 2,000, 3,000, 4,000, 5,000 mm or more in length.

An interior lumen of a nerve conduit can comprise one or more materials having neurogenic, neuroinductive, and/or neuroconductive properties. Neurogenic materials can cause cells to differentiate into cells with the properties of mature neurons. Neuroinductive materials promote the development of neurons, by e.g., triggering a signaling pathway. Neuroconductive materials can facilitate transfer of electrical signals between nerves. A neuroconductive material has high electrical conductivity (e.g., about electrical conductivity of 1×10⁵ S/m or higher).

### Decellularized Human Placental Connective Tissue Matrix (DHPCTM)

In an aspect, DHPCTM is present in the lumen of nerve conduit. In an aspect, the DHPCTM is allogenic. DHPCTM can provide a native extracellular matrix (ECM) for cell attachment. ECM is mostly a protein-based structure in tissues that acts as a skeleton to support and hold cells in place. In an aspect, DHPCTM contains no residual growth factors, cytokines, cells, cell debris, or nucleic acids. DHPCTM can be isolated from human placentas. In an aspect DHPCTM is derived from a chorionic plate of a placenta. In an aspect, DHPCTM has anti-inflammatory properties that can prevent necrosis at the nerve repair site.

DHPCTM can suppress expression of pro-inflammatory mediators, including cytokines and matrix-degrading enzymes. DHPCTM can reduce expression of TNF-α, IL-1β, and IL-6; decrease matrix metalloproteinases (MMPs) associated with chronic inflammation and tissue breakdown; and preserve normal cellular phenotype under inflammatory stress. DHPCTM can also support a transition from M1 (pro-inflammatory) macrophages to M2 (anti-inflammatory, pro-repair) macrophages, which can resolve chronic inflammation by re-establishing a regenerative immune environment.

Nerve conduits have traditionally been made of collagen or synthetic bioresorbable polymers. Type I collagen conduits are bioabsorbable and semi-permeable (e.g., NeuraGen® Nerve Guide (Integra LifeSciences); NeuroFlex®/NeuroMatrix® (Collagen Matrix/Regenity); Neurawrap®). However, the performance of these types of conduits declines beyond short gaps and are not equivalent to autograft for long or motor nerve defects. Synthetic bioresorbable polymer conduits are made of polyglycolic acid (PGA), polycaprolactone (PCL), or PLGA copolymers (e.g., Neurotube® (Synovis); Neurolac® (Polyganics). However, foreign body reactions can occur with these types of nerve conduits. The instant nerve conduits comprising DHPCTM can have lower nerve morbidity and lower immune reactions (e.g., about 5, 10, 25, 50, 75, 80, 90% lower) than nerve conduits made of collagen or synthetic bioresorbable polymers.

Decellularized ECM can be derived from placental tissue using any suitable method. For example, freezing the placental tissues can be used to cause cell rupture and separation of tissue layers. Decellularization can involve mechanical and/or chemical methods to remove cells from tissues and retain the intact ECM structures. In one aspect, DHPCTM can be prepared from frozen placentas as described by Bhatia et al., Placenta Derived Adherent Cell (PDAC) Interaction and Response on Extracellular Matrix Isolated from Human Placenta. Wounds. 2008 Feb.; 20(2): 29-36. Briefly, frozen placentas can be thawed in sodium chloride and then ground up and washed in sodium chloride. From here, the placental matter can be washed in detergent (e.g., 1% SDS or deoxycholic acid) and treated with 0.1-0.5 N sodium hydroxide for about 3-24 hours and then rinsed with PBS to neutralize the pH. The material can be formed into a flowable paste or particulate matter. DHPCTM can comprise extracellular matrix with high collagen content, e.g., Type I, Type III, and Type IV, elastin, and glycosaminoglycans. Fibronectin and laminin can be present where sodium hydroxide is not used in the preparation of the DHPCTM. DHPCTM can mimic native ECM and create a reparative environment to promote ECM remodeling, cell migration, proliferation, and differentiation. An example of DHPCTM is INTERFYL®, Celularity, Florham Park, NJ.

### Adipose Tissue and Bone Marrow Aspirate

In an aspect a lumen of a nerve conduit can comprise adipose tissue (e.g., micronized adipose tissue), bone marrow aspirate, or a combination thereof. In an aspect, the adipose tissue or bone marrow aspirate can be an autogenic graft. That is, the adipose tissue or bone marrow aspirate is derived from the same patient to whom the material will later be re-introduced. In an aspect, the adipose tissue or bone marrow aspirate is an allogenic graft. In an aspect, the adipose tissue or bone marrow aspirate is harvested from the patient to be treated, micronized, and washed prior to placement within the nerve conduit. Washing the adipose tissue or bone marrow aspirate can reduce any inflammatory reaction in the patient.

Micronized adipose tissue is a processed form of fat tissue where the fat cells have been broken down into fragments through, for example, a chemical or mechanical process. Micronization can concentrate beneficial cellular components like adipose-derived stem cells (ASCs) within the tissue. Micronized adipose tissue can comprise cellular components, such as ASCs, mesenchymal stem cells (MSCs), pericytes, fibroblasts, blood cells, endothelial progenitor cells, and immune cells; extracellular matrices (ECMs); and cytokines.

Micronized adipose tissue particles can be from about 0.1 to about 1.0 mm in diameter, for example about 0.1, 0.2, 0.3, 0.4, 0.5, 0.6, 0.7, 0.8, 0.9, or about 1.0 mm or more in diameter. In an aspect micronized adipose tissue particles are about 1.0, 0.9, 0.8, 0.7, 0.6, 0.5, 0.4., 0.3, 0.2, 0.1 mm or less in diameter. In an aspect micronized adipose tissue particles range from about 0.1 to about 1 mm in diameter, from about 0.2 to about 0.9 mm in diameter, from about 0.2 to about 0.5 mm in diameter, from about 0.4 to about 0.8 mm in diameter, or from about 0.6 to about 0.8 mm in diameter.

Micronized adipose tissue can be prepared using, for example Arthrex's AUTOPOSE™ adipose harvesting system or other suitable method. Briefly, adipose tissue can be harvested from a patient and injected into an AUTOPOSE™ double-syringe system through the Luer transfer port. The adipose tissue can be washed twice by injecting sterile saline into the syringe, inverting for 10 seconds, allowing the tissue to gravimetrically separate for 3 min, and then decanting completely. After washing, the outer flanges of the AUTOPOSE™ syringe can be pushed to micro fragment the adipose tissue through an 800 μm filter into the inner syringe. Other systems for producing micronized adipose tissue are commercially available.

Bone marrow aspirate is the liquid part of the soft tissue inside a subject's bones that contains a mixture of white blood cells (including progenitor cells, lymphocytes, and granulocytes) and platelets, as well as growth factors and proteins. Bone marrow aspirate can be isolated using, e.g., harvesting needles from Arthrex, Naples, FL. Techniques for bone marrow harvesting are disclosed in, for example, Allahabadi et al.,

Bone Marrow Aspirate Concentrate Harvest Techniques for the Sports Medicine Surgeon, Arthroscopy Techniques, Vol. 13(2024 ).

### Nerve Allograft or Autograft

In an aspect nerve allograft or autograft material can be added to the lumen of the nerve conduit. For example, about 0.1, 0.5, 1.0, 1.5, 2.0, 3.0, 4.0, 5.0, 10.0, 15.0 mL or more can be used to fill the lumen of a nerve conduit depending upon the size of the nerve conduit. In an aspect, nerve autograft material can be micronized nerve tissue derived from the damaged nerve to be repaired (e.g., the stumps of a bisected nerve or crushed nerve tissue). In an aspect, nerve allograft or autograft material can be one or more intact portions of a nerve that are about 1.0, 1.25, 1.5, 1.75, 2.0, 3.0, 4.0, 5.0, 10, 25, 50, 75, 100, 250, 500, 750, 1,000, 1,250, 1,500, 1,750, 2,000 mm or more in length. In an aspect, the nerve allograft or autograft material can be placed longitudinally in a lumen of a nerve conduit.

In an aspect, an allograft or autograft nerve graft can be acellular. A nerve graft can be decellularized and processed, resulting in a graft comprising natural structural pathways to guide axon regeneration. Decellularization and processing and of the nerve allograft can remove or reduce axonal and myelin debris. Decellularization can be done mechanically, chemically, enzymatically, or a combination thereof. Further processing can remove material and molecules that can potentially elicit immune responses in the recipient or that could result in infection.

### Additional Components

In an aspect, additional components can be added to the lumen of a nerve conduit. For example, nerve growth stimulating agents such as neurotrophic, chemotactic, or mitogenic agents that can stimulate axonal growth directly or indirectly can be added to a nerve conduit. Examples of these agents include insulin-like growth factor 1, insulin-like growth factor 2, platelet derived growth factors (e.g., PDGF-A, PDGF-B, PDGF-C, and PDGF-D), fibroblast growth factors (e.g. FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, or Keratinocyte growth factor-2), transforming growth factor-α, transforming growth factor-β, brain derived neurotrophic factor, ciliary neurotrophic factor, leukemia inhibitory factor, interleukin-6, oncostatin M, prolactin, neurturin, and glial cell line-derived neurotrophic factor. Nerve growth stimulating agents can also comprise whole cells such as Schwann cells, endothelial cells, fibroblasts, genetically altered cells, or combinations thereof. In an aspect, any one of these materials can be present in the lumen at about 0.0001, 0.001, 0.01, 0.1, 1.0, 2, 3, 4, 5, 10, 15, 20% or more of the total material in the nerve conduit.

### Nerve Repair Device

An aspect provides a nerve repair device comprising a nerve conduit having a proximal end, a distal end, an exterior surface, and an interior surface defining a lumen. The lumen can comprise a mixture of allogeneic decellularized human placental connective tissue matrix (DHPCTM). The lumen can further comprise adipose tissue, such as micronized adipose tissue, and/or bone marrow aspirate. In an aspect, nerve allograft or autograft can be added to the lumen of the device.

In an aspect, nerve allograft can provide neuroconductive and neuroinductive properties. Nerve conduits can provide neuroconductive properties. A nerve conduit by itself does not have neurogenic properties. Adipose tissue, bone marrow aspirate, and mesenchymal stem cells (present in bone marrow aspirate and adipose tissue, or independently added to the lumen of the nerve conduit) can provide neurogenic properties and in some cases neuroconductive properties. Decellularized human placental connective tissue matrix can provide neuroinductive properties by providing cellular induction of mesenchymal stem cells to Schwann cells.

The adipose tissue and/or bone marrow aspirate can be autograft or allograft adipose tissue and/or bone marrow aspirate. The mixture in the lumen can further comprise nerve autograft or allograft material. The lumen can further comprise insulin-like growth factor 1, insulin-like growth factor 2, platelet derived growth factors (e.g., PDGF-A, PDGF-B, PDGF-C, and PDGF-D), fibroblast growth factors (e.g. FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, or Keratinocyte growth factor-2), transforming growth factor-α, transforming growth factor-β, brain derived neurotrophic factor, ciliary neurotrophic factor, leukemia inhibitory factor, interleukin-6, oncostatin M, prolactin, neurturin, glial cell line-derived neurotrophic factor, Schwann cells, endothelial cells, fibroblasts, genetically altered cells, or combinations thereof.

Nerves that can be repaired include, for example, abdominal aortic plexus, abducens nerve, accessory nerve, accessory obturator nerve, Alderman's nerve, anococcygeal nerve, ansa cervicalis, anterior interosseous nerve, anterior superior alveolar nerve, Auerbach's plexus, auriculotemporal nerve, axillary nerve, brachial plexus, buccal nerve, cardiac plexus, cavernous plexus, celiac ganglion, cervical plexus, chorda tympani, ciliary ganglion, coccygeal nerve, cochlear nerve, common fibular nerve, common palmar digital nerve, common peroneal nerve, cranial nerve, cutaneous nerve, deep fibular nerve, deep peroneal nerve, deep petrosal nerve, deep temporal nerves, dorsal scapular nerve, esophageal plexus, ethmoidal nerve, external laryngeal nerve, external nasal nerve, facial nerve, femoral nerve, frontal nerve, gastric plexuses, geniculate ganglion, genitofemoral nerve, glossopharyngeal nerve, greater auricular nerve, greater occipital nerve, greater petrosal nerve, hepatic plexus, hypoglossal nerve, iliohypogastric nerve, ilioinguinal nerve, inferior alveolar nerve, inferior anal nerve, inferior cardiac nerve, inferior cervical ganglion, inferior gluteal nerve, inferior hypogastric plexus, inferior mesenteric plexus, inferior palpebral nerve, infraorbital nerve, infraorbital plexus, infratrochlear nerve, intercostal nerves, intercostobrachial nerve, intermediate cutaneous nerve, internal carotid plexus, internal laryngeal nerve, interneuron, jugular ganglion, lacrimal nerve, lateral cord, lateral pectoral nerve, lateral plantar nerve, lateral pterygoid nerve, lesser occipital nerve, lingual nerve, long ciliary nerve, long thoracic nerve, lower subscapular nerve, lumbar nerve, lumbar plexus, lumbar splanchnic nerve, lumboinguinal nerve, lumbosacral plexus, lumbosacral trunk, mandibular nerve, masseteric nerve, maxillary nerve, medial cord, medial cutaneous nerve, medial pectoral nerve, medial plantar nerve, medial pterygoid nerve, median nerve, Meissner's plexus, mental nerve, middle meningeal nerve, motor nerve, musculocutaneous nerve, mylohyoid nerve, nasociliary nerve, nasopalatine nerve, nerve of pterygoid canal, nerve to obturator internus, nerve to quadratus femoris, nerve to the piriformis, nerve to the stapedius, nerve to the subclavius, nervus intermedius, nervus spinosus, nodose ganglion, obturator nerve, occipital nerve, oculomotor nerve, olfactory nerve, ophthalmic nerve, optic nerve, optic ganglion, ovarian plexus, palatine nerve, pancreatic plexus, patellar plexus, pelvic splanchnic nerves, perforating cutaneous nerve, perineal nerve, petrous ganglion, pharyngeal nerve, pharyngeal plexus, phrenic nerve, phrenic plexus, posterior auricular nerve, posterior cord, posterior scrotal nerve, posterior superior alveolar nerve, prostatic plexus (nervous), pterygopalatine ganglion, pudendal nerve, pudendal plexus, radial nerve, recurrent laryngeal nerve, renal plexus, sacral plexus, sacral splanchnic nerves, saphenous nerve, sciatic nerve, semilunar ganglion, sensory nerve, short ciliary nerve, sphenopalatine nerve, splenic plexus, subcostal nerve, submandibular ganglion, suboccipital nerve, superficial fibular nerve, superficial peroneal nerve, superior cardiac nerve, superior cervical ganglion, superior gluteal nerve, superior hypogastric plexus, superior labial nerve, superior laryngeal nerve, superior mesenteric plexus, superior rectal plexus, supraclavicular nerve, supraorbital nerve, suprarenal plexus, suprascapular nerve, supratrochlear nerve, sural nerve, sympathetic nerve, sympathetic trunk, thoracic aortic plexus, thoracic splanchnic nerve, thoraco-abdominal nerve, thoracodorsal nerve, tibial nerve, transverse cervical nerve, trigeminal nerve, trochlear nerve, tympanic nerve, ulnar nerve, upper subscapular nerve, uterovaginal plexus, vagus nerve, ventral ramus, vesical nervous plexus, vestibular nerve, vestibulocochlear nerve, zygomatic nerve, zygomaticofacial nerve, and zygomaticotemporal nerve.

### Methods of Preparation of a Nerve Repair Device

In an aspect, a method of preparing a nerve repair device is provided. The methods can be performed in vitro, ex vivo, or in vivo. DHPCTM can be placed into a lumen of a nerve conduit having a proximal end, a distal end, an exterior surface, and an interior surface defining the lumen; to form a filled nerve conduit. In an aspect, adipose tissue and/or bone marrow aspirate also be added to the lumen. Adipose tissue and/or bone marrow aspirate can be harvested from a subject. The subject can be the patient to be treated or an adipose tissue or bone marrow aspirate donor. In an aspect, the adipose tissue and/or bone marrow aspirate can be micronized and washed prior to mixing. The adipose tissue and/or bone marrow aspirate can be mixed with DHPCTM. The ratio of adipose tissue and/or bone marrow aspirate to DHPCTM can be about 1:1, 1:2, 1:3, 1:4, 1:5, 5:1, 4:1, 3:1, 2:1 or any other suitable ratio. The DHPCTM or DHPCTM and adipose tissue and/or bone marrow aspirate mixture can be placed into a lumen of a nerve conduit having a proximal end, a distal end, an exterior surface, and an interior surface defining the lumen; to form a filled nerve conduit. Other components can be added to the lumen, including, for example, allograft or autograft nerve tissue, insulin-like growth factor 1, insulin-like growth factor 2, platelet derived growth factors (e.g., PDGF-A, PDGF-B, PDGF-C, and PDGF-D), fibroblast growth factors (e.g. FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, or Keratinocyte growth factor-2), transforming growth factor-α, transforming growth factor-β, brain derived neurotrophic factor, ciliary neurotrophic factor, leukemia inhibitory factor, interleukin-6, oncostatin M, prolactin, neurturin, glial cell line-derived neurotrophic factor, Schwann cells, endothelial cells, fibroblasts, genetically altered cells, or combinations thereof.

### Methods of Treatment

An aspect provides methods of nerve repair. The methods comprise providing a nerve repair device as described herein and placing the proximal end of the nerve conduit in contact with a first portion of a nerve and the distal end of the nerve conduit in contact with a second portion of the nerve such that the first and second portions of the nerve are each positioned in contact with the distal and proximal ends or within the lumen at each of the distal and proximal ends. The first portion of the nerve can be secured to the proximal end of the nerve conduit and the second portion of the nerve can be secured to the distal end of the nerve conduit. Sutures can be used to secure the nerve portions to the nerve conduit.

Placing the proximal end of the nerve conduit of the nerve device in contact with a first portion of the nerve and the distal end of the nerve conduit in contact with a second portion of the nerve can comprise loading the nerve conduit into a needle or catheter and injecting the nerve conduit into a tissue. In an aspect, a nerve repair device can be administered by inserting (e.g., injecting or placing) the nerve repair device to a region in need.

In an aspect, the first portion of the nerve and the second portion of the nerve can be opposite ends of a fully or partially transected nerve. In an aspect, the first portion of the nerve and the second portion of the nerve can include proximal and distal portions of an injured but still intact nerve (e.g., a crushed nerve). A nerve injury can comprise the complete or partial transection of the nerve or the compression or crushing of a nerve or nerve segment. A nerve injury can comprise an acute crush injury, a repetitive crush injury, or an acute compression injury or a repetitive compression injury.

Other nerve injuries that can be treated include, for example, an injury to a peripheral nerve of a patient. An injury to a peripheral nerve can be caused by e.g., a peripheral neuropathy, trauma, cancer, surgery, a congenital anomaly, a disease or condition (e.g., amyotrophic lateral sclerosis, carpal tunnel syndrome), amputation, and/or complete or partial removal of an organ, tumor, or tissue, radiation therapy, chemotherapy, metabolic/endocrine complications including diabetes, inflammatory and autoimmune diseases, vitamin deficiencies including vitamins B6 and B12, infectious diseases, toxic causes, exposure to organic metals and heavy metals, and side effects of drugs.

In an aspect, the injury can be hand, finger, foot, or toe injuries, such as injuries caused by sharp objects, traumatic or surgical injuries of any type involving the upper or lower extremity resulting in acute or delayed loss of the nerve function, or nerve deficit injuries following nerve tumor removal.

In an aspect, devices and methods disclosed herein can be used to repair nerves during breast re-construction surgery.

Methods can comprise treating a spinal cord injury (e.g., partial or complete transection of the spinal cord, crush injury of the spinal cord, or compression of the spinal cord) or traumatic brain injury (e.g., penetrating or non-penetrating brain injuries).

In an aspect, a nerve repair device can be used to treat a damaged or lost nerve having a length of about 0.1, about 0.5, about 1.0, about 1.5, about 2.0, about 2.5, about 3.0, about 3.5, about 4.0, about 4.5, about 5.0, about 6.0, about 7.0, about 8.0, about 9.0, about 10.0, or more centimeters (cm) in length.

A patient can be a human, a non-human primate, equine, bovine, sheep, canine, feline, porcine, lapine, cameline, caprine, or ovine.

In an aspect, fibrin sealant can be placed on, over, or around the nerve repair device after it is placed into a subject. Fibrin sealant is a solution of concentrated fibrinogen (e.g., bovine or human fibrinogen) that is activated by the addition of thrombin (e.g., bovine or human thrombin) and calcium chloride. When activated, the thrombin converts fibrinogen into insoluble fibrin. Examples of fibrin sealants include Tisseel, Tachosil, and Artiss, Baxter, Westlake Village, CA; Evicel and Evarrest, Ethicon/J&J, Somerville, NJ; Vitagel, Orthovita/Stryker, Malvern PA.

In an aspect, autologous thrombin enhanced serum (from, e.g., a Thrombinator™ system (Arthrex, Naples, FL)) can be placed on, over, or around the nerve repair device after it is placed into a subject. The Thrombinator™ System can produce an autologous thrombin rich serum. An autologous thrombin rich serum comprises about 5 to about 20 U/mL of thrombin activity. An autologous thrombin rich serum comprises more thrombin activity than the starting fluid. In an aspect, a 2-stage process can be used to produce autologous, thrombin-enhanced serum. In the first stage, the Thrombinator™ system activates the coagulation cascade within the initial aliquot of autologous blood-derived fluid injected into the device. Blood-derived fluid can be, e.g., whole blood, autologous conditioned plasma), or platelet-poor plasma. The activated device can be treated with a second aliquot of a blood-derived fluid to produce the autologous thrombin rich serum. In an aspect, an autologous thrombin rich serum also comprises enriched levels of Il-1Ra, IL-1β, TGF-β1, PDGF-AB, and VEGF as compared to the starting fluid.

### Kits

Kits are provided herein that provide materials for making and delivering a nerve repair device. Kits can comprise one or more components. For example, a kit can comprise a system for harvesting adipose tissue and/or bone marrow aspirate including a needle and syringe. The system can also comprise components for washing and micronizing the adipose tissue. For example a syringe or container comprising a filter through which the tissue can be forced through and washed. An example is the adipose harvesting AUTOPOSE™ system of Arthrex, Naples FL.

A kit can additionally comprise decellularized human placental connective tissue matrix (DHPCTM). The DHPCTM can be particulate matter or flowable matter. An example is INTERFYL® human connective tissue matrix, Celularity, Florham Park, NJ.

A kit can additionally comprise a container to mix micronized adipose tissue and/or bone marrow aspirate with the DHPCTM. Additional components can be added to the micronized adipose tissue and/or bone marrow aspirate and DHPCTM and can be included in a kit (e.g., allograft or autograft nerve tissue, insulin-like growth factor 1, insulin-like growth factor 2, platelet derived growth factors (e.g., PDGF-A, PDGF-B, PDGF-C, and PDGF-D), fibroblast growth factors (e.g. FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, or Keratinocyte growth factor-2), transforming growth factor-α, transforming growth factor-β, brain derived neurotrophic factor, ciliary neurotrophic factor, leukemia inhibitory factor, interleukin-6, oncostatin M, prolactin, neurturin, glial cell line-derived neurotrophic factor, Schwann cells, endothelial cells, fibroblasts, genetically altered cells, or combinations thereof).

A kit can further comprise buffers or diluents (e.g., saline) for mixing the components.

A kit can additionally comprise a nerve conduit having a proximal end, a distal end, an exterior surface, and an interior surface defining the lumen. One or more nerve conduits of varying sizes can be included in a kit. In an aspect a nerve conduit can be cut to any desired size. In an aspect, a kit provides one or more nerve conduits of about 2 mm to about 50 mm. In an aspect, a kit provides one or more nerve conduits of about 2 mm to about 90 mm.

A kit can additionally comprise an insertion catheter for filling the nerve conduit with the micronized adipose tissue and/or bone marrow aspirate and/or DHPCTM (and any other components).

A kit can additionally comprise sutures, suture needles, and anchors.

In an aspect, the kit can be stable at room temperature and does not require refrigeration. In an aspect, a kit can be stable at 0, 3, 5, 10, 15, 20, 22.5, 25, 30 °C or more for 30, 60, 90, 120, 360 or more days.

In an aspect, a method of treating an injury to a nerve is provided where a proximal and a distal end of the nerve are separated by a gap. The method can comprise introducing a nerve repair device as described herein into the gap. A nerve repair device, when placed to a gap between two portions of a nerve, covers at least about 50 percent, or at least about 75 percent, or at least about 80 percent, or at least about 90 percent, of the gap between the proximal and distal nerve ends. In an aspect, the gap is at least about 0.5 cm to about 1 cm. In some aspects the gap is at least about 2, 3, 4, 5, 6 cm or more.

The compositions and methods set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The aspects illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by aspects and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

1. A nerve repair device comprising a nerve conduit having a proximal end, a distal end, an exterior surface, and an interior surface defining a lumen, the lumen comprising decellularized human placental connective tissue matrix (DHPCTM).

2. A nerve repair device of claim 1, wherein the lumen further comprises adipose tissue, bone marrow aspirate, or both adipose tissue and bone marrow aspirate.

3. A nerve repair device of claim 2, wherein the adipose tissue is micronized adipose tissue.

4. The nerve repair device of claim 1, wherein the lumen further comprises nerve autograft material or nerve allograft material.

5. The nerve repair device of claim 3, wherein the micronized adipose tissue and/or bone marrow aspirate is autograft micronized adipose tissue and/or autograft bone marrow aspirate.

6. (canceled)

7. (canceled)

8. The nerve repair device of claim 1, wherein the nerve conduit is comprised of one or more of collagen, poly(lactic-co-glycolic acid) (PLGA), silk, polytetrafluoroethylene (PTFE), polydimethylsiloxane (PDMS), silicone, a salicylic acid-based polymer, a salicylsalicylic acid-based polymer, a difluorophenyl-salicylic acid-based polymer, collagen, fibrin, fibronectin, alginate, gelatin, keratin, thrombin, poly-glycolic acid, poly-(lactic acid), poly-caprolactone, poly-(L-lactic acid), poly(3-hydroxybutyric acid), poly((bis(hydroxyethyl) terephthalate-ethyl phosphoester/terephthaloyl chloride), poly(2-hydroxyethyl methacrylate) (PHEMA), a co-polymer of PHEMA and methyl methacrylate, and poly(acrylonitrile-co-methylacrylate).

9. (canceled)

10. (canceled)

11. (canceled)

12. The nerve repair device of claim 1, wherein the lumen further comprises insulin-like growth factor 1, insulin-like growth factor 2, PDGF-A, PDGF-B, PDGF-C, PDGF-D), fibroblast growth factor (FGF)1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, Keratinocyte growth factor-2, transforming growth factor-α, transforming growth factor-β, brain derived neurotrophic factor, ciliary neurotrophic factor, leukemia inhibitory factor, interleukin-6, oncostatin M, prolactin, neurturin, glial cell line-derived neurotrophic factor, Schwann cells, endothelial cells, fibroblasts, genetically altered cells, or combinations thereof.

13. (canceled)

14. A method of nerve repair comprising placing a proximal end of a nerve conduit in contact with a first portion of the nerve and a distal end of the nerve conduit in contact with a second portion of the nerve such that the first and second portions of the nerve are each positioned in contact with the distal and proximal ends of the nerve conduit or within a lumen at each of the distal and proximal ends, wherein the nerve conduit comprises a proximal end, a distal end, an exterior surface, and an interior surface defining the lumen, the lumen comprising decellularized human placental connective tissue matrix (DHPCTM).

15. The method of claim 14, wherein the lumen further comprises adipose tissue, bone marrow aspirate, or both adipose tissue and bone marrow aspirate.

16. (canceled)

17. The method of claim 14, wherein the first portion of the nerve is secured to the proximal end of the nerve conduit and the second portion of the nerve is secured to the distal end of the nerve conduit.

18. The method of claim 14, wherein the first portion of the nerve and the second portion of the nerve are opposite ends of a fully or partially transected nerve.

19. The method of claim 14, wherein the first portion of the nerve and the second portion of the nerve are proximal and distal portions of an injured but still intact nerve.

20. (canceled)

21. (canceled)

22. The method of claim 14, wherein placing the proximal end of the nerve conduit of the nerve device in contact with a first portion of the nerve and the distal end of the nerve conduit in contact with a second portion of the nerve comprises loading the nerve conduit into a needle or catheter and injecting the nerve conduit into a tissue.

23. The method of claim 14, wherein the lumen further comprises nerve autograft material, nerve, allograft material, insulin-like growth factor 1, insulin-like growth factor 2, PDGF-A, PDGF-B, PDGF-C, PDGF-D), fibroblast growth factor (FGF)1, FGF 2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, Keratinocyte growth factor-2, transforming growth factor-α, transforming growth factor-β, brain derived neurotrophic factor, ciliary neurotrophic factor, leukemia inhibitory factor, interleukin-6, oncostatin M, prolactin, neurturin, glial cell line-derived neurotrophic factor, Schwann cells, endothelial cells, fibroblasts, genetically altered cells, or combinations thereof.

24. The method of claim 14, further comprising contacting the nerve repair device with fibrin adhesive, autologous, thrombin-enhanced serum, or a combination thereof.

25. (canceled)

26. A method of nerve repair in a subject comprising:
harvesting adipose tissue and/or bone marrow aspirate from the subject and mixing the adipose tissue and/or bone marrow aspirate with DHPCTM to form a mixture;
placing the mixture into a lumen of a nerve conduit having a proximal end, a distal end, an exterior surface, and an interior surface defining the lumen;
placing the proximal end of the nerve conduit in contact with a first portion of a nerve and the distal end of the nerve conduit in contact with a second portion of the nerve such that the first and second portions of the nerve are each positioned in contact with the distal and proximal ends of the nerve conduit or within the lumen at each of the distal and proximal ends of the nerve conduit.

27. (canceled)

28. The method of claim 26, wherein the lumen further comprises nerve autograft material, nerve, allograft material, insulin-like growth factor 1, insulin-like growth factor 2, PDGF-A, PDGF-B, PDGF-C, PDGF-D), fibroblast growth factor (FGF)1, FGF 2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, Keratinocyte growth factor-2, transforming growth factor-α, transforming growth factor-β, brain derived neurotrophic factor, ciliary neurotrophic factor, leukemia inhibitory factor, interleukin-6, oncostatin M, prolactin, neurturin, glial cell line-derived neurotrophic factor, Schwann cells, endothelial cells, fibroblasts, genetically altered cells, or combinations thereof.

29. The method of claim 26, further comprising contacting the nerve repair device with fibrin adhesive, autologous, thrombin-enhanced serum, or a combination thereof.

30. (canceled)

31. A method of preparing the nerve repair device of claim 1, comprising harvesting adipose tissue and/or bone marrow aspirate from a subject and mixing the adipose tissue and/or bone marrow aspirate with DHPCTM to form a mixture and placing the mixture into a lumen of a nerve conduit having a proximal end, a distal end, an exterior surface, and an interior surface defining the lumen; to form a filled nerve conduit.

32. (canceled)

33. The method of claim 31, wherein the lumen further comprises nerve autograft material, nerve, allograft material, insulin-like growth factor 1, insulin-like growth factor 2, PDGF-A, PDGF-B, PDGF-C, PDGF-D), fibroblast growth factor (FGF)1, FGF 2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, Keratinocyte growth factor-2, transforming growth factor-α, transforming growth factor-β, brain derived neurotrophic factor, ciliary neurotrophic factor, leukemia inhibitory factor, interleukin-6, oncostatin M, prolactin, neurturin, glial cell line-derived neurotrophic factor, Schwann cells, endothelial cells, fibroblasts, genetically altered cells, or combinations thereof.

34.-40. (canceled)
